(19) United States
(12) Patent Application Publication
(10) Pub. No.: US 2009/0169633 A1
(43) Pub. Date: Jul. 2, 2009

(54) Oral particle including pseudoephedrine hydrochloride and cetirizine dihydrochloride

(76) Inventor: Ta-Ping Liao, Chia Yi City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/003,687

(22) Filed: Dec. 31, 2007

(51) Int. Cl. A61K 9/14; A61K 31/137

(52) U.S. Cl. 424/490; 514/646

(57) ABSTRACT

The present invention relates to an oral particle including pseudoephedrine hydrochloride and cetirizine dihydrochloride, which primarily includes a nucleus having a diameter ranging 25˜40 mesh, a pseudoephedrine-hydrochloride layer coated outside the nucleus with a coating solution composed of pseudoephedrine hydrochloride, a binder, a lubricant and pure water/alcohol, a release-control layer coated outside the pseudoephedrine-hydrochloride layer, and a cetirizine-dihydrochloride layer coated outside the release-control layer with a coating solution composed of cetirizine dihydrochloride, a binder, a lubricant and pure water/alcohol. Accordingly, by distributing pseudoephedrine hydrochloride and cetirizine dihydrochloride into hundreds of the particles and controlling the dissolution rate with the release-control layer, the particles can perform good absorption efficiency, and quick, stable and long-term medicinal effect.

### BACKGROUND OF THE INVENTION

1. Field of the Invention

The present invention relates to an oral particle including pseudoephedrine hydrochloride and cetirizine dihydrochloride, and particularly to an oral particle performs good absorption efficiency, quick and stable response effect and long-term medicinal effect by distributing pseudoephedrine hydrochloride and cetirizine dihydrochloride among hundreds of the particles and controlling the dissolution rate with the release-control layer.

2. Related Prior Arts

The oral medicine including pseudoephedrine hydrochloride and cetirizine dihydrochloride is generally used to treat Seasonal and perennial allergic rhinitis, for example, nasal congestion, sneeze, runny nose and nose and eyes itch. Currently, the market oral medicine including pseudoephedrine hydrochloride and cetirizine dihydrochloride is in the form of a tablet, for example, CIRRUS®, which primarily contains a pseudoephedrine-hydrochloride layer and a thin cetirizine-dihydrochloride layer outside the pseudoephedrine-hydrochloride layer. For such a tablet, enough dosage of pseudoephedrine hydrochloride and cetirizine dihydrochloride is required to achieve a therapeutic effect, which therefore results to a larger volume.

When one takes the tablet including pseudoephedrine hydrochloride and cetirizine dihydrochloride, bioavailability is limited and absorption is slow as the single tablet has only limited surface area for acting with gastric acid. In addition, the cetirizine-dihydrochloride layer coated outside of the pseudoephedrine-hydrochloride layer is hydrophilic and will be completely dissolved while being taken and only pseudoephedrine hydrochloride is remained to directly act with gastric acid to form a sticky gel being slowly released. Because of smaller surface areas and varying dissolubility with individualities, the tablets can not release their effective components in a constant and stable rate and therefore their effects for treating the above diseases are influenced.

### SUMMARY OF THE INVENTION

The object and effect of the present invention is to provide oral particles including pseudoephedrine hydrochloride and cetirizine dihydrochloride, which can perform good absorption efficiency, and quick, stable and long-term medicinal effect by distributing pseudoephedrine hydrochloride and cetirizine dihydrochloride in hundreds of the particles and controlling dissolution rate of pseudoephedrine hydrochloride with a release-control layer.

Accordingly, the oral particle of the present invention primarily includes a nucleus having a diameter ranging 25˜40 mesh, a pseudoephedrine-hydrochloride layer coated outside the nucleus and including pseudoephedrine hydrochloride, a binder, a lubricant and pure water/alcohol, a release-control layer coated outside the pseudoephedrine-hydrochloride layer and including a matrix, talc powder and pure water, and a cetirizine-dihydrochloride layer coated outside the release-control layer and including cetirizine dihydrochloride, a binder, a lubricant and pure water/alcohol.

The oral medicine of the present invention can be manufactured as different pharmaceutical types. A suitable type is to include hundreds of the oral particles aforementioned in a capsule made from an animal or a plant, so that each capsule may contain 120 mg of active pseudoephedrine hydrochloride and 5 mg of active cetirizine dihydrochloride.

In the present invention, the nucleus is preferably made from sugar which comprises 65˜95 wt. % of sucrose and pharmaceutical inert or neutral starch.

The above pseudoephedrine-hydrochloride layer can be formed with a coating solution composed of pseudoephedrine hydrochloride, a binder and a lubricant dissolved in a solvent system including pure water and alcohol. In the coating solution, the pure water/alcohol has a weight ratio ranging from 80/20 to 20/80, and preferably about 50/50; and the solid content, i.e., pseudoephedrine hydrochloride, the binder and the lubricant in the coating solution, ranges 10˜30 wt. %, and preferably 20 wt. %. The alcohol is preferably ethanol.

Components of the above release-control layer include methyl acrylate and talc powder which are diluted in pure water.

The above cetirizine-dihydrochloride layer can be formed with a coating solution composed of cetirizine dihydrochloride, a binder and a lubricant in a solvent system including pure water and alcohol. In the coating solution, the pure water/alcohol has a weight ratio ranging from 80/20 to 20/80, and preferably about 50/50; and the solid content, i.e., cetirizine dihydrochloride, the binder and the lubricant in the coating solution, ranges 15˜35 wt. %, and preferably 27 wt. %. The alcohol is preferably ethanol.

### BRIEF DESCRIPTION OF THE DRAWINGS

FIG. 1 shows the average concentrations of cetirizine dihydrochloride in blood of volunteers taking the particles of the present invention (Hiros S.R.M Cap) and the market tablets (CIRRUS®).

FIG. 2 shows the average concentrations of pseudoephedrine hydrochloride in blood of six volunteers taking the particles of the present invention (Hiros S.R.M Cap and the market tablets (CIRRUS®).

FIG. 3 is a chart showing Relative Bioavailability of Cetirizine Dehydrochloride; and

FIG. 4 is a chart showing Relative Bioavailability of Pseudoephedrine Hydrochloride.

### ATTACHMENTS

Attachment 1 compares the relative bioavailability of cetirizine dihydrochloride in blood of volunteers taking the particles of the present invention (Hiros S.R.M Cap) and the market tables (CIRRUS®).

Attachment 2 compares the relative bioavailability of pseudoephedrine hydrochloride in blood of volunteers taking the particles of the present invention (Hiros S.R.M Cap) and the market tables (CIRRUS®).

### DETAILED DESCRIPTION OF THE PREFERRED EMBODIMENTS

To more clearly explain features, objects and effects the present invention, preferred embodiments are described accompanied with figures.

In the present invention, the oral particle including pseudoephedrine hydrochloride and cetirizine dihydrochloride includes an inner core capable of controlling and releasing pseudoephedrine hydrochloride and an outer layer.

The inner core includes:
(a) a nucleus:
(b) a pseudoephedrine-hydrochloride layer formed by being coated with a coating solution composed of pseudoephedrine hydrochloride, a binder, a lubricant and pure water/alcohol;
(c) a release-control layer formed by being coated with a coating solution composed of a matrix selected from either methyl acrylate or ethyl cellulose, talc powder and pure water; and
the above layers are outwardly formed layer by layer.

The outer layer coated outside the release-control layer is substantially a cetirizine-dihydrochloride layer which is formed by being coated with a coating solution composed of nonsedative antihisamine cetirizine dihydrochloride, a binder, a lubricant and pure water/alcohol.

Preferably, the oral particle including cetirizine-dihydrochloride and pseudoephedrine-hydrochloride includes:

### 1. Inner Core

### (a) Nucleus

The nucleus has a diameter ranging 25˜40 mesh and a weight percentage ranges 17˜27 wt. % on the basis of the oral particle, and preferably about 20 wt. %. The nucleus is preferably made from sugar composed of 65˜95 wt. % of sucrose and pharmaceutical inert or neutral starch.

### (b) Pseudoephedrine-hydrochloride Layer

The pseudoephedrine-hydrochloride layer has a weight percentage ranges 40˜65 wt. % on the basis of the oral particle, and preferably 54 wt. %. The coating solution includes pseudoephedrine hydrochloride, binder and lubricant in a solvent system including pure water and alcohol. The weight ratio of pure water/alcohol ranges 80/20˜20/80, and preferably about 50/50. The alcohol is preferably ethanol. The solid content, i.e., pseudoephedrine hydrochloride, binder and lubricant in the coating solution, ranges 10˜30 wt. % on the basis of the coating solution, and preferably 20 wt. %. The lubricant includes at least one of talc powder and stearic acid; and the binder includes at least one of hydroxyl propyl methyl cellulose, hydroxyl propyl cellulose and povidone.

### (c) Release-Control Layer

The release-control layer has a weight percentage ranges 15˜30 wt. % on the basis of the oral particle, and preferably 26 wt. %. The release-control layer includes 40˜60 wt. % of methyl acrylate, 5˜10 wt. % of talc powder and 35˜50 wt. % of pure water for diluting the other components. Preferably, the contents of methyl acrylate, the talc powder and pure water are 50 wt. %, 7 wt. % and 43 wt. %, respectively.

### 2. Outer Layer

The tablet film, substantially a cetirizine-dihydrochloride layer, has a weight percentage ranges 3˜8 wt. % on the basis of the oral particle, and preferably 4.5 wt. %. The coating solution includes cetirizine dihydrochloride, the binder and the lubricant in a solvent system including pure water and alcohol. In the coating solution, contents of cetirizine dihydrochloride, the binder and the lubricant are 20˜40 wt. %, 8˜20 wt. % and 30˜60 wt. %, respectively. The weight ratio of pure water/alcohol ranges 80/20˜20/80, and preferably 50/50. The alcohol is preferably ethanol. The solid content, i.e., cetirizine dihydrochloride, the binder and the lubricant in the coating solution, ranges 15˜35 wt. %, and preferably 27 wt. %.

A plurality of the particles can be packaged or processed as various medicines, for example, a capsule containing hundreds of the particles. The capsule can be made from an animal or a plant. Preferably, each capsule includes 120 mg of active pseudoephedrine hydrochloride and 5 mg of active cetirizine dihydrochloride.

The nucleus having a diameter about 25˜40 mesh is suitable for operation in a rotary-spray granulator. The spraying speed should be carefully adjusted to avoid drying of the coating solution at a low speed and congregation at a high speed. To overcome these problems, a lower initial speed can be applied and then the speed is increased when the particles are growing larger.

The air flowing rate can be controlled with an outlet valve and optimized according to circulation of the particles. A lower flowing rate will reduce amount of the pellets; and a higher rate may hinder the particles circulating. In the preferred embodiments, the valve is adjusted to about 50% of the maximum at beginning and then gradually to about 60%.

During the coating process, temperature of the introduced air is about 40° C.˜50° C., and rotary speed of the disk is about 60˜80 rpm.

After the coating process, a drying process for about 20˜30 minutes is necessary to remove additional solvents.

### EXAMPLE 1

(a) Preparing the Coating Solution for the pseudoephedrine-hydrochloride Layer

In a container, pure water (250 kg) and ethanol (250 kg) are added and mixed. Then pseudoephedrine hydrochloride (92.4 kg), stearic acid (23.1 kg), talc powder (7.55 kg) and hydroxyl propyl cellulose (4.2 kg) are added into the container to obtain the coating solution for the pseudoephedrine-hydrochloride layer. The coating solution is filtered with a sieve of 80-mesh.

### (b) Preparing the Coating Solution for the Control-Release Layer

Talc powder (12.4 kg) is mixed with pure water (60 kg) which is blended in a container. Then the mixture is filtered with a sieve of 100-mesh. In another container, methyl acrylate (66 kg) is added and blended with a mixer, then the filtered liquid is slowly added and mixed well to obtain the coating solution for the control-release layer. The coating solution is filtered with a sieve of 100-mesh.

(c) Preparing the Coating Solution for the cetirizine-dihydrochloride Layer

In a container, pure water (15.4 kg) and ethanol (15.4 kg) are blended, and hydroxyl propyl methyl cellulose (1540 g) is then add and completely dissolved. Then cetirizine dihydrochloride (3850 g) and talc powders (6160 g) are added and mixed to obtain the coating solution for the cetirizine-dihydrochloride layer. The coating solution is filtered with a sieve of 100-mesh.

(d) Loading the pseudoephedrine-hydrochloride Layer

In a rotating granulator equipped with a disk of 1 m diameter, sugar nuclei (47.6 kg, 25˜30 mesh or 590˜710 μm) are placed. Temperature and flowing rate of the inlet gas is about 45° C.˜55° C. and about 30˜35 m³/min, flowing rate of the outlet gas is about 35˜38 m³/min, rotary speed of the disk is about 60˜80 rpm, pressure of the injector is about 5.0˜6.0 kg/cm², and the loading is gradually increased from 500 g/min to 800 g/min. After complete loading, the particles are dried with 50˜60° C. air for 20 minutes.

### (e) Coating the Control-Release Layer

The dried particles are continued coated. Temperature of the inlet gas is about 35˜40° C. Flowing rates of the inlet and outlet gas are remained. Rotary speed of the disk is about 80˜90 rpm. Pressure of the injector is about 6.0 kg/cm². The loading is about 500˜700 g/min.

(f) Coating the cetirizine-dihydrochloride Layer

The particles are continued coated. Temperature of the inlet gas is about 45˜55° C. Flowing rates of the inlet and outlet gas are remained. Rotary speed of the disk is about 60˜80 rpm. Pressure of the injector is about 5.0 kg/cm². The loading is about 600˜800 g/min.

### (g) Drying

After complete the coating process and stop spraying, rotary speed of the disk is reduced to 30 rpm, temperature of the inlet gas is increased to 55˜60° C. for 30 minutes for drying the particles. The particles are then cooled to about 25° C. and discharged into a proper container.

### (h) Screening

The particles are screened with a multiple sieve of 14 mesh and 30 mesh. The particles are classified into “no good” (over 14 mesh), “good” (14˜30 mesh), and “no good” (below 30 mesh).

### (i) Encapsulating

The “good” particles are encapsulated into No. 0 capsules by an automatic machine. Each capsule contains about 334 mg of particles, i.e., about 5 mg of cetirizine dihydrochloride and 120 mg of pseudoephedrine-hydrochloride.

### EXAMPLE 2

(a) Loading the pseudoephedrine-hydrochloride Layer

Four equivalent amounts of binder solutions are prepared by adding hydroxyl propyl cellulose into ethanol which is blended in a container. Total amount of ethanol is 26250 g, and total amount of hydroxyl propyl cellulose is 1050 g.

Then pseudoephedrine hydrochloride (92.4 kg), stearic acid (23.1 kg) and talc powder (7.55 kg) are crushed and separated into four equivalent amounts of powders.

One selected from four equivalent amounts of sugar nuclei (47.6 kg, 25˜30 mesh or 590˜710 μm) is placed in a centrifugal granulating coater (CF-1000) which is operated at 120˜150 rpm and 1000 NL/min˜1500 NL/min of gas flowing rate. An equivalent amount of the binder solution is slowly added at a rate of 250 g/min until 300 g. The powders are added for loading drugs until all powders are added and unloaded. The unloaded particles are then dried in a dryer at 50° C. for 4 hours.

### (b) Preparing the Coating Solution for the Control-Release Layer

Talc powder (12.4 kg) is mixed with pure water (60 kg) which is blended in a container. Then the mixture is filtered with a sieve of 100-mesh. In another container, methyl acrylate (66 kg) is added and blended with a mixer, then the filtered liquid is slowly added and mixed well to obtain the coating solution for the control-release layer. The coating solution is filtered with a sieve of 100-mesh.

(c) Preparing the Coating Solution for the cetirizine-dihydrochloride Layer

In a container, pure water (15.4 kg) and ethanol (15.4 kg) are blended, and hydroxyl propyl methyl cellulose (1540 g) is then add and completely dissolved. Then cetirizine dihydrochloride (3850 g) and talc powders (6160 g) are added and mixed to obtain the coating solution for the cetirizine-dihydrochloride layer. The coating solution is filtered with a sieve of 100-mesh.

### (d) Coating the Control-Release Layer

In a rotating granulator equipped with a disk of 1 m diameter, the dried particles are continued coated. Temperature of the inlet gas is about 35˜40° C. Flowing rates of the inlet and outlet gas are 30˜35 m³/min and 35˜38 m³/min, respectively. Rotary speed of the disk is about 80˜90 rpm. Pressure of the injector is about 6.0 kg/cm². The loading is about 500˜700 g/min.

(e) Coating the cetirizine-dihydrochloride Layer

The particles are continued coated. Temperature of the inlet gas is about 45˜55° C. Flowing rates of the inlet and outlet gas are remained. Rotary speed of the disk is about 60˜80 rpm. Pressure of the injector is about 5.0 kg/cm². The loading is about 600˜800 g/min.

### (f) Drying

After complete the coating process and stop spraying, rotary speed of the disk is reduced to 30 rpm, temperature of the inlet gas is increased to 55˜60° C. for 30 minutes for drying the particles. The particles are then cooled to about 25° C. and discharged into a proper container.

### (g) Screening

The particles are screened with a multiple sieve of 14 mesh and 30 mesh. The particles are classified into “no good” (over 14 mesh), “good” (14˜30 mesh), and “no good” (below 30 mesh).

### (h) Encapsulating

The “good” particles are encapsulated into No. 0 capsules by an automatic machine. Each capsule contains about 334 mg of particles, i.e., about 5 mg of cetirizine dihydrochloride and 120 mg of pseudoephedrine hydrochloride.

According to the present invention, the particles including pseudoephedrine hydrochloride and cetirizine dihydrochloride can promote bioavailability as the solubility of cetirizine dihydrochloride is increased and pseudoephedrine hydrochloride is stable and can be released for a long time.

### Clinic Experiments:

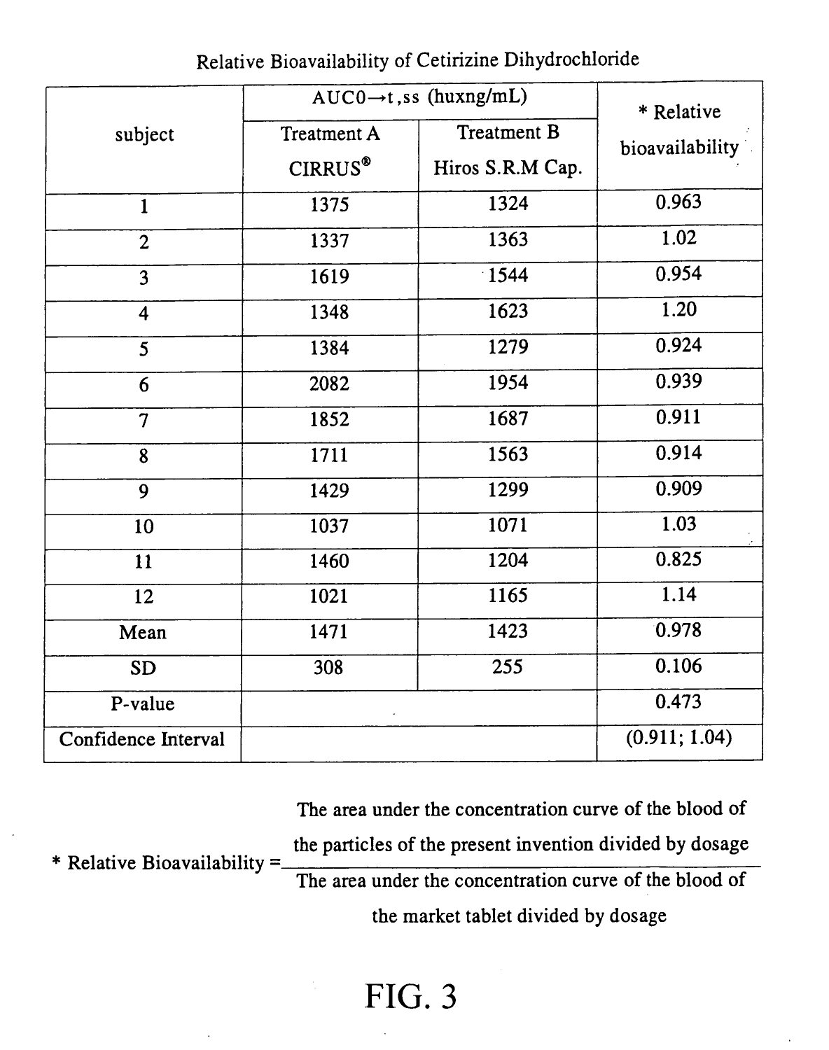

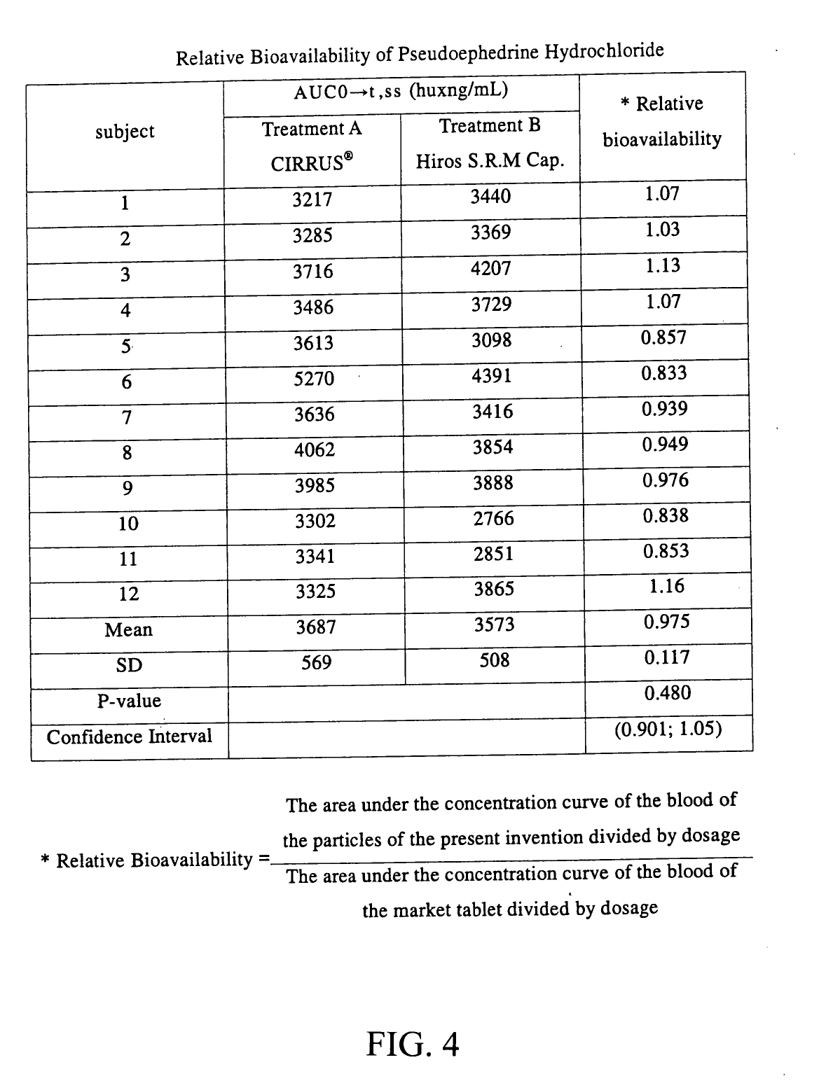

1. The average excretion half time of cetirizine dihydrochloride in human body is 7.56±1.00 hours, and that of pseudoephedrine hydrochloride is 4.76±0.44 hours. The capsule including 334 mg of the particles of the present invention, i.e., 5 mg of cetirizine dihydrochloride and 120 mg of pseudoephedrine hydrochloride is compared with the market tablet CIRRUS® each including 5 mg of cetirizine dihydrochloride and 120 mg of pseudoephedrine hydrochloride.
2. Twelve healthy volunteers (subjects) are orally applied with the capsule medicine of the present invention (Hiros S.R.M Cap) and the market tablets (CIRRUS®). Their bloods are sampled and analyzed. Attachment 1 compares areas (AUC0→τ,ss) under concentration curves of cetirizine dihydrochloride in the blood of the volunteers taking the particles of the present invention (Hiros S.R.M Cap) and the market tablets (CIRRUS®). In FIG. 3, an average of the areas (AUC0→τ,ss) under concentration curves of the blood of the volunteers taking the particles of the present invention (Hiros S.R.M Cap) is 1423 (huxng/mL), and the standard deviation (SD) is 255; and an average of the areas (AUC0→τ,ss) under concentration curves of the blood of the volunteers taking the market tablets (CIRRUS®) is 1471 (huxng/mL), and the standard deviation (SD) is 308. FIG. 4 compares areas (AUC0→τ,ss) under concentration curves of pseudoephedrine hydrochloride in the blood of the volunteers taking the particles of the present invention (Hiros S.R.M Cap) and the market tablets (CIRRUS®). In FIG. 4, an average of the areas (AUC0→τ,ss) under concentration curves of the blood of the volunteers taking the particles of the present invention (Hiros S.R.M Cap) is 3573 (huxng/mL), and the standard deviation (SD) is 508; and an average of the areas (AUC0→τ,ss) under concentration curves of the blood of the volunteers taking the market tablets (CIRRUS®) is 3687 (huxng/mL), and the standard deviation (SD) is 569.

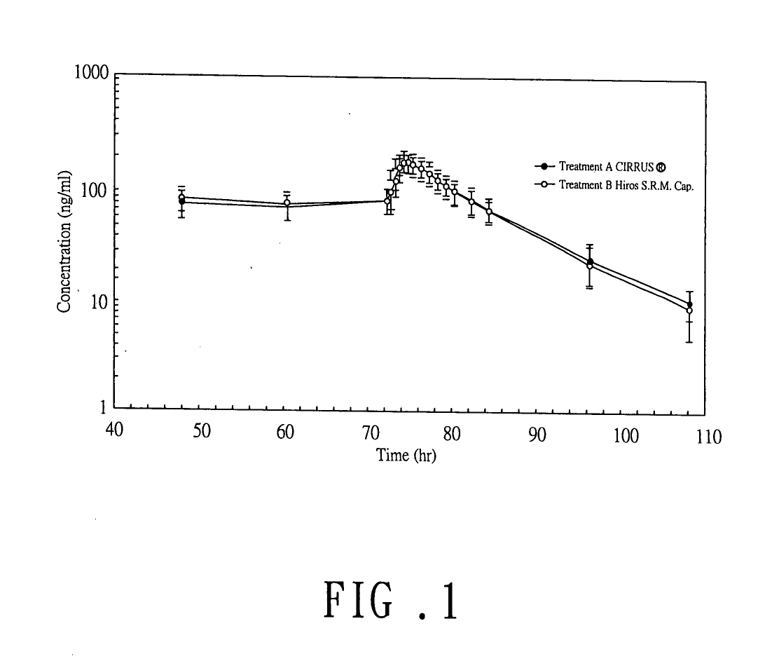

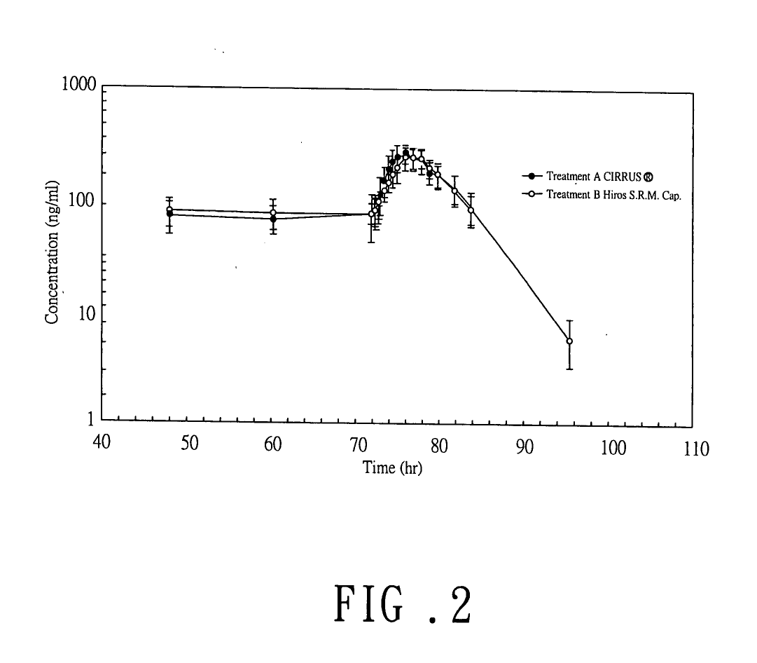

According to the above statistic data and standard deviations of the areas (AUC0→τ,ss) under concentration curves of the blood, the market tablets have a larger standard deviation than the particles of the present invention. That is, the particles of the present invention perform better stability than the market tablets, or the particles of the present invention are more stable and have longer release time than the market tablets. Please also refer to FIGS. 1 and 2.

According to the above, by distributing pseudoephedrine hydrochloride and cetirizine dihydrochloride into hundreds of the particles and controlling the dissolution rate with the release-control layer, the particles can perform good absorption efficiency, and quick, stable and long-term medicinal effect.

What is claimed is:

1. An oral particle including pseudoephedrine hydrochloride and cetirizine dihydrochloride, at least comprising an inner core capable of controlling and releasing pseudoephedrine hydrochloride and an outer layer; wherein
the inner core comprises:
(a) a nucleus having a diameter ranging about 25˜40 mesh;
(b) a pseudoephedrine-hydrochloride layer formed by coating with a coating solution composed of pseudoephedrine hydrochloride, a binder, a lubricant and pure water/alcohol;
(c) a release-control layer formed by coating with a coating solution composed of a matrix including either methyl acrylate or ethyl cellulose, talc powder and pure water; and
the outer layer is substantially a cetirizine-dihydrochloride layer which is coated outside the release-control layer with a coating solution composed of nonsedative antihisamine cetirizine dihydrochloride, a binder, a lubricant and pure water/alcohol.

2. The oral particle including pseudoephedrine hydrochloride and cetirizine dihydrochloride as claimed in claim 1, wherein the nucleus is made from sugar which comprises 65˜95 wt. % of sucrose and pharmaceutical inert or neutral starch.

3. The oral particle including pseudoephedrine hydrochloride and cetirizine dihydrochloride as claimed in claim 1, wherein the weight ratio of the pure water/alcohol in the coating solution for the pseudoephedrine-hydrochloride layer ranges from 80/20 to 20/80.

4. The oral particle including pseudoephedrine hydrochloride and cetirizine dihydrochloride of claim 3, wherein the weight ratio of the pure water/alcohol in the coating solution for the pseudoephedrine-hydrochloride layer is about 50/50.

5. The oral particle including pseudoephedrine hydrochloride and cetirizine dihydrochloride as claimed in claim 1, wherein the alcohol is ethanol.

6. The oral particle including pseudoephedrine hydrochloride and cetirizine dihydrochloride as claimed in claim 1, wherein the solid content of pseudoephedrine hydrochloride, the binder and the lubricant in the coating solution for the pseudoephedrine-hydrochloride layer ranges 10˜30 wt. %.

7. The oral particle including pseudoephedrine hydrochloride and cetirizine dihydrochloride of claim 6, wherein the solid content of pseudoephedrine hydrochloride, the binder and the lubricant in the coating solution for the cetirizine dihydrochloride layer is about 20 wt. %.

8. The oral particle including pseudoephedrine hydrochloride and cetirizine dihydrochloride as claimed in claim 1, wherein the lubricant includes at least one of talc powder and stearic acid.

9. The oral particle including pseudoephedrine hydrochloride and cetirizine dihydrochloride as claimed in claim 1, wherein the binder includes at least one of hydroxyl propyl methyl cellulose, hydroxyl propyl cellulose and povidone.

10. The oral particle including pseudoephedrine hydrochloride and cetirizine dihydrochloride as claimed in claim 1, wherein the coating solution for the release-control layer comprises 40˜60 wt. % of methyl acrylate and 5˜10 wt. % of talc powder which both are diluted in 35˜50 wt. % of pure water.

11. The oral particle including pseudoephedrine hydrochloride and cetirizine dihydrochloride as claimed in claim 10, wherein the coating solution for the release-control layer comprises about 50 wt. % of methyl acrylate and about 7 wt. % of talc powder which both are diluted in about 43 wt. % of pure water.

12. The oral particle including pseudoephedrine hydrochloride and cetirizine dihydrochloride as claimed in claim 1, wherein the contents of cetirizine dihydrochloride, the binder and the lubricant in the cetirizine-dihydrochloride layer range 20˜40 wt. %, 8˜20 wt. % and 30˜60 wt. %, respectively.

13. The oral particle including pseudoephedrine hydrochloride and cetirizine dihydrochloride as claimed in claim 1, wherein the weight ratio of the pure water/alcohol in the coating solution for the cetirizine-dihydrochloride layer ranges 80/20˜20/80.

14. The oral particle including pseudoephedrine hydrochloride and cetirizine dihydrochloride as claimed in claim 13, wherein the weight ratio of the pure water/alcohol in the coating solution for the cetirizine-dihydrochloride layer is about 50/50.

15. The oral particle including pseudoephedrine hydrochloride and cetirizine dihydrochloride as claimed in claim 1, wherein the solid content of cetirizine dihydrochloride, the binder, the lubricant and the pure water/alcohol in the coating solution for the cetirizine-dihydrochloride layer ranges 15˜35 wt. %.

16. The oral particle including pseudoephedrine hydrochloride and cetirizine dihydrochloride as claimed in claim 15, wherein the solid content of cetirizine dihydrochloride, the binder, the lubricant and the pure water/alcohol in the coating solution for the cetirizine-dihydrochloride layer is about 27 wt. %.

17. An oral medicine including pseudoephedrine hydrochloride and cetirizine dihydrochloride, comprising:
a plurality of the oral particles including pseudoephedrine hydrochloride and cetirizine dihydrochloride as claimed in claim 1; and
a capsule made from an animal or a plant for packing the oral particles therein so that the oral medicine contains 120 mg of active pseudoephedrine hydrochloride and 5 mg of active cetirizine dihydrochloride.

18. The oral particle including pseudoephedrine hydrochloride and cetirizine dihydrochloride as claimed in claim 1, which comprises 17˜27 wt. % of the nucleus, 40˜65 wt. % of the pseudoephedrine-hydrochloride layer, 15˜30 wt. % of the release-control layer and 3˜8 wt. % of the outer layer, all based on the oral particle.

19. The oral particle including pseudoephedrine hydrochloride and cetirizine dihydrochloride as claimed in claim 18, which comprises about 20 wt. % of the nucleus, about 54 wt. % of the pseudoephedrine-hydrochloride layer, 26 wt. % of the release-control layer and about 4.5 wt. % of the outer layer, all based on the oral particle.
